(19) United States
(12) Patent Application Publication
(10) Pub. No.: US 2015/0217016 A1
(43) Pub. Date: Aug. 6, 2015

(54) METHODS, SYSTEMS AND DEVICES COMPRISING SCENTED ARTIFICIAL TREES

(71) Applicant: EnviroScent, Inc., Atlanta, GA (US)

(72) Inventors: Nicholas D. McKay, Atanta, GA (US); Emrie Sherwood, Atlanta, GA (US); Jeffrey S. Sherwood, Ellijay, GA (US)

(73) Assignee: EnviroScent, Inc., Atlanta, GA (US)

(21) Appl. No.: 14/426,095

(22) PCT Filed: Sep. 4, 2013
(86) PCT No.: PCT/US2013/058052

§ 371 Date: Mar. 4, 2015

Related U.S. Application Data
(60) Provisional application No. 61/696,684, filed on Sep. 4, 2012.

(51) Int. Cl. A61L 9/04; A61L 9/03; A61L 9/12

(52) U.S. Cl. CPC A61L 9/042; A61L 9/12; A61L 9/122; A61L 9/03

(57) ABSTRACT

In an aspect, an artificial tree comprising a trunk and a series of branches extending outwardly from the trunk further comprises an olfactory-active substance. In embodiments, the olfactory-active substance is provided by use of a reservoir containing the olfactory-active substance which can be provided to at least a portion of the tree by capillary action, gravitational force, or by an electric pump. In embodiments, an olfactory-active substance is integral to the material components of the tree which have been suitably treated, coated or amalgamated with one or more olfactory-active substances.

### RELATED APPLICATIONS

The present invention claims the priority of and benefit of the filing date of U.S. Provisional Patent Application Ser. No. 61/696,684, filed Sep. 4, 2012, which is herein incorporated in its entirety.

### BACKGROUND

Artificial trees are a well-known invention and they are sold around the world for a variety of purposes. Many of the currently produced artificial trees are noted for their realistic appearance and texture. However, one aspect of realism where artificial trees are deficient is their scent. Decorative live plants, in particular Christmas trees, are often fondly noted for their pleasing aroma and scent. As such, there has been and continues to be a need for the development of artificial trees capable of giving off a lifelike scent.

Accordingly, there have been attempts to use aerosol sprays, scented candles, incense, potpourri, and free standing air fresheners, among other devices, to recapitulate the scent of an artificial tree.

This desire to reproduce the smell of a tree as an added feature of a lifelike artificial tree has led those skilled in the art to develop a variety of methods for reaching this end, and there have been multiple attempts in the prior art to produce an artificial tree encompassing some kind of device for emitting a scent or aroma. Examples of such devices are known to those in the art. However, many of these examples have proved to be limited in commercial appeal for a variety of reasons.

Thus, it would be highly desirable for an improved system to fill the unmet need in this segment of the artificial tree market.

### SUMMARY

The invention disclosed herein comprises methods, systems and devices for introducing a scent into the ambient air through a physical support comprising an artificial tree. The tree comprises a trunk with an upper portion having a plurality of outwardly extending branches, and a lower portion generally free of outwardly extending branches. The trunk may be generally columnar in shape and generally vertically oriented along a longitudinal axis, for instance, an artificial Christmas tree or artificial palm tree would both be exemplary illustrations. An artificial tree may comprise a plurality of decorations on the tree including but not limited to decorative lights or fiber optics, decorative balls or tinsel, artificial or real flowers, or artificial animals.

Various means of emitting the scent from the tree are disclosed herein, including but not limited to the use of a capillary action system feeding from a reservoir at the base to the tree; a gravity feed system feeding from the top of the tree; constructing the tree from material components that have been suitably amalgamated with a fragrance or treated so as to emit a scent; an internal or hidden housing system for the placement of fragrance emitting devices; a pump system for transporting fragrance into a porous matrix that may include the artificial leaves, needles, or branches as well as an interior portion of the tree; one or more scented strands or cables, which may or may not have electrical connections, that may line or cover various portions of the tree; scented attachment elements that can be placed on to or inside the tree; scented accessories for the tree that may hold fragrance-emitting devices; fragrant packaging for the artificial tree; and a warming scenting device that may be on a timer.

### FIGURES

FIG. 1 shows an exemplary embodiment of the present invention.

### DETAILED DESCRIPTION

The present invention provides methods, systems and devices for introducing a scent into the ambient air through a physical support comprising an artificial tree.

The invention comprises methods, systems and devices for introducing a scent into the ambient air through a physical support comprising an artificial tree. The tree comprises a trunk with an upper portion having a plurality of outwardly extending branches, and a lower portion generally free of outwardly extending branches. The trunk may be generally columnar in shape and generally vertically oriented along a longitudinal axis, for instance, an artificial Christmas tree or artificial palm tree would both be exemplary illustrations. An artificial tree may comprise a plurality of decorations on the tree including but not limited to decorative lights or fiber optics, decorative balls or tinsel, artificial or real flowers, or artificial animals.

The tree as defined herein includes an artificial representation of any kind of tree including but not limited to Christmas tree, evergreen trees, palm trees, magnolia trees, cherry blossom trees, bonsai trees, fruit trees, ficus trees, bamboo trees, and any other tropical, evergreen or deciduous trees. Tree may also refer to other decorative members of the Plantae kingdom including but not limited to flowers and flower arrangements, bushes, and shrubs.

A tree comprises a trunk, from which a plurality of branches are outwardly extending. The plurality of branches may generally extend out from the upper portion of the trunk, while the lower portion may be generally devoid of branches. The trunk may be generally columnar in shape and generally vertically oriented along a longitudinal axis. The trunk may be hollow and comprise one or more apertures such as slits or holes such that ambient air can pass freely through them. A trunk may comprise a longitudinally oriented tube with apertures through at least a portion of the tube, and also comprise a covering for the apertures, whether inside the trunk tube or outside the trunk tube, which may be slid or rotatably moved over one or more apertures in the trunk tube. Opening the apertures by moving the covering away from the apertures would allow a fragrance within the trunk tube to be released from the trunk tube, and closing the apertures by moving the covering over at least a portion of the openings would inhibit the release of the fragrance from the trunk tube. Alternatively, the trunk may be solid. Optionally, a tree may comprise lights.

An aspect of the present invention comprises adding one or more olfactory-active substances to one or more substrates, such as a matrix material made from pulp, paper, plastic or other suitable matrix material, or the olfactory-active substances or compositions to the structural component(s) of an artificial tree, which may be made from a matrix material disclosed herein, or the olfactory-active substance may be added to both a matrix material and a structural component of a tree. The olfactory-active substance may be moved from one location to another within or on the artificial tree by use of a reservoir or pump, wherein the olfactory-active substance may be dispersed throughout the physical support or structural elements of an artificial tree by means of capillary action, gravity, compressive forces or other means.

Olfactory-active substances include but are not limited to, fragrances, repellants, odor-eliminating compounds, aromatherapy compounds, natural oils, water-based scents, odor neutralizing compounds, and cyclodextrins, and such terms may be used interchangeably, and include olfactory-active compositions or fragrance compositions, or olfactory-active compounds. As used herein, “olfactory-active substance” refers to any compound, mixture or suspension of compounds that are odorous, or compounds mixture or suspension of compounds that cancel or neutralize odorous compounds, such as any compound or combination of compounds that would produce a positive or negative olfactory sense response in a living being that is capable of responding to olfactory compounds, or that reduces or eliminates such olfactory responses. A fragrance of the present invention comprises an aroma or odorous compound, mixture or suspension of compounds that is capable of producing an olfactory response in a living being capable of responding to olfactory compounds, and may be referred to herein as odorant, aroma, or fragrance. An olfactory-active composition as used herein comprises one or more olfactory-active substances and is generally a composition that has a smell or odor, which may be volatile, which may be transported to the olfactory system of a human or animal, and is generally provided in a sufficiently high concentration so that it will interact with one or more olfactory receptors. A fragrance composition may include one or more than one of the fragrance characteristics, including topnotes, midnotes or heart, and the drydown or base notes. An olfactory composition may comprise other diluents or additives, such as solvents or preservatives.

An embodiment of the present invention comprises an olfactory-active substance, such as a fragrance, an artificial tree to serve as a physical support, a reservoir at the base of said tree for holding the olfactory-active substance, and a capillary system for transporting the olfactory-active substance throughout the tree. The interior or exterior of the trunk may be lined with a plurality of capillaries for the purpose of drawing the olfactory-active substance up from the reservoir and throughout the tree. The capillaries may be contained entirely within a hollow trunk or attached to the outside of a solid trunk. The capillaries may be comprised of a suitable size, substance, and matrix such that the olfactory-active substance may be drawn up from the reservoir through the trunk and throughout the rest of the tree by capillary action while maintaining the ability to emit the scent of the olfactory-active substance.

The capillaries may be arranged such that one end hangs down into or is resting in the reservoir at the bottom of the tree. The reservoir may be incorporated into the tree stand or base or it may be a stand-alone element.

The capillaries may be comprised of a suitable size, substance, and matrix such that the olfactory-active substance may be drawn up through the trunk by capillary action while maintaining the ability to emit the scent of the olfactory-active substance. Such substances are known and may include but are not limited to felt, paper, pulp or other matrices with suitable wicking or capillary properties.

The capillaries may be provided individually or in groups of two or more, and may be held in a receptacle having apertures to allow the passage of fragrance molecules. Capillaries may be provided in any shape, for example, as a rod or a shaped rod or like a web. Capillaries may optionally comprise dyes, coating compositions, holder elements or attachment elements.

The capillaries of the present invention comprise a structural component which is made from a matrix material composition. A structural component may be coated, dyed, or further treated in forming an article of the present invention.

The present invention comprises compositions used in making a capillary. A structural component may comprise one or more matrix material compositions. A matrix material composition may comprise plastics, porous materials, natural and synthetic pulp compositions, or pulp compositions combined with other products, including but not limited to paper, cellulose, cellulose acetate, pulp lap, cotton linters, biological plant-derived materials (from living plants), synthesized pulp compositions, mixed pulps, felts and other cloths. A matrix material composition may comprise additives, materials that provide strength, rigidity or structure to a matrix material composition. For example, a composition may comprise nanomaterials as additives.

The matrix material used within the capillary may be a variety of wicking materials including pulps and felts of different densities and porosities as well as those materials incorporated herein by reference from published US patent application US 2011/0262377 A1.

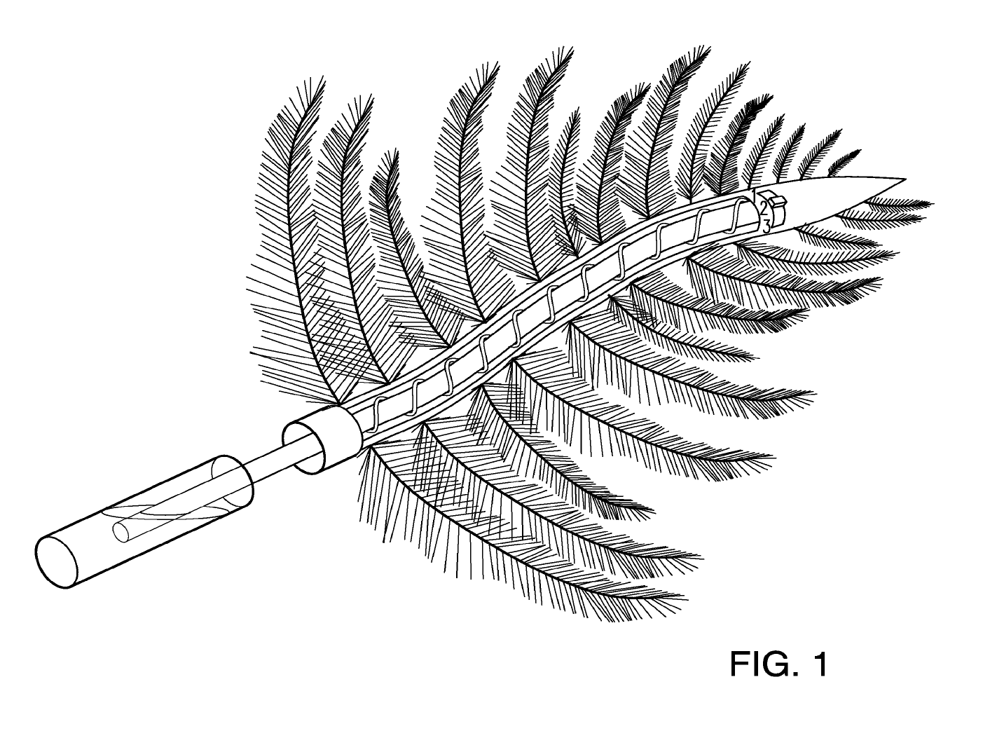

See FIG. 1, which shows an exemplary artificial tree. The trunk of the tree is hollow and contains a wicking element. The wicking element may be a unitary wick of a single structure that can move the fragrance from the reservoir through the interior of the trunk and release the fragrance near the top of the tree through a vent. The vent is capable of being opened to several positions to allow for greater or lesser release of fragrance from the interior of the hollow trunk. The wicking element may comprise a plurality of capillary tubes that move the fragrance composition from the reservoir at the bottom of the tree to the vent. The reservoir may be refilled with a fragrance composition or may be a single-use disposable container.

An embodiment of the present invention comprises an olfactory-active substance, such as a fragrance, an artificial tree to serve as a physical support, a reservoir at the base of said tree for holding the olfactory-active substance, and a capillary system for transporting the olfactory-active substance throughout the tree. The interior or exterior of the trunk may be lined with a plurality of capillaries for the purpose of drawing the olfactory-active substance up from the reservoir wherein the network of capillaries inside and outside of the trunk divide to travel through or on the branches of the tree. The capillaries may be fed into hollow needles, or if the capillary is of suitable material, it may be shaped and dyed in order to simulate a needle. The capillaries may be comprised of a suitable size, substance, and matrix such that the olfactory-active substance may be drawn up from the reservoir through the trunk and throughout the rest of the tree by capillary action while maintaining the ability to emit the scent of the olfactory-active substance. The capillaries may be porous throughout or in a portion of the body of the capillary so that fragrance may be released from the capillaries, or a capillary may end in a releasing element where the fragrance is released, such as a microreservoir, sponge or valve that releases milliliter amounts of fragrance.

The capillaries may be arranged such that one end hangs down into or is resting in the reservoir at the bottom of the tree. The reservoir may be incorporated into the tree stand or base or it may be a stand-alone element.

The capillaries may be comprised of a suitable size, substance, and matrix such that the olfactory-active substance may be drawn up through the trunk by capillary action while maintaining the ability to emit the scent of the olfactory-active substance. Such substances are known and may include but are not limited to felt, paper, pulp, plastic or other matrices with suitable wicking or capillary properties.

The capillaries may be provided individually or in groups of two or more, and may be held in a receptacle having apertures to allow the passage of fragrance molecules. Capillaries may be provided in any shape, for example, as a rod or a shaped rod or like a web. Capillaries may optionally comprise dyes, coating compositions, holder elements or attachment elements.

The capillaries of the present invention comprise a structural component of the artificial tree and may be made from a matrix material composition. A structural component may be coated, dyed, or further treated in forming an artificial tree of the present invention.

The present invention comprises compositions used in making a capillary. A structural component may comprise one or more matrix material compositions. A matrix material composition may comprise natural and synthetic pulp compositions, plastics, absorbent materials, pulp compositions combined with other products, including but not limited to paper, cellulose, cellulose acetate, pulp lap, cotton linters, biological plant-derived materials (from living plants), synthesized pulp compositions, mixed pulps, felts and other cloths. A matrix material composition may comprise additives, materials that provide strength, rigidity or structure to a matrix material composition. For example, a composition may comprise nanomaterials as additives.

A matrix material used within the capillary may be a variety of wicking materials including pulps and felts of different densities and porosities as well as those materials incorporated herein by reference from published US patent application US 2011/0262377 A1.

An embodiment comprises an olfactory-active substance, such as a fragrance, an artificial tree to serve as a physical support, a reservoir at the top of the tree for holding the olfactory-active substance, and a series of transport elements connected to the reservoir that are capable of moving the olfactory-active substance within and throughout the tree. A transport element may be a capillary as described herein. The interior or exterior of the trunk may be lined with a plurality of transportation elements, such as but not limited to tubes or strips of wicking material, for the purpose of moving the olfactory-active substance down from the reservoir at the top of the tree and throughout the trunk of the tree. Transport elements may be comprised of a suitable size, substance, and matrix such that the olfactory-active substance may be drawn down through the trunk by gravity while maintaining the ability to emit the scent of the olfactory-active substance.

A transport element may be provided individually or in groups of two or more, and may be inside or outside of the trunk. Transport elements may be provided in any shape, for example, as a tube or a sleeve. Transport elements may optionally comprise dyes, coating compositions, holder elements or attachment elements as necessary.

A transport element of the present invention comprises a structural component suitable for transporting the olfactory-active substance from the reservoir to one or more distal points on the tree, whether the reservoir is located at the bottom of the tree, the top of the tree or at some other location in or on the tree. Transport elements may be comprised of tubes with minuscule openings or pores or micro-pores or mesh-like sections for the purpose of allowing fragrance molecules to emit from the olfactory active substance within the transport element. Transport elements may be comprised of permeable material, such as felt, paper, string, or cloth that allows fragrance to seep out of the olfactory-active substance therein or the transportation elements may be comprised of any other suitably porous material or pattern for the purpose of emitting fragrance. Alternatively, the transport elements may comprise closed tubing or similar structure that is only open at the termini. Release of olfactory-active substance from the termini may be controlled by the size of the hole that is present, a valve, or a fragrance-emitting device capable of holding the olfactory-active substance and emitting the associated fragrance. The fragrance emitting device may be but is not limited to a sponge or sponge-like material, a pulp or paper matrix, or any other suitable substrate for the purpose of controlling release from the transport elements while emitting fragrance. The fragrance-emitting devices, which may be made from a matrix material composition, may contain within them an olfactory-active substance.

The present invention comprises compositions used in making a fragrance-emitting device. A structural component may comprise one or more matrix material compositions. A matrix material composition may comprise natural and synthetic pulp compositions, or pulp compositions combined with other products, including but not limited to paper, cellulose, cellulose acetate, pulp lap, cotton linters, biological plant-derived materials (from living plants), synthesized pulp compositions, and mixed pulps. A matrix material composition may comprise additives, materials that provide strength, rigidity or structure to a matrix material composition. For example, a composition may comprise nanomaterials as additives. A fragrance-emitting device of the present invention may be referred to as a pulp article, indicating that the article comprises a matrix material composition which was made from a pulp composition of the present invention.

The matrix material used within the fragrance-emitting device may be a variety of materials including pulps, papers, sponges, and felts of different densities and porosities as well as those materials incorporated herein by reference from published US patent application US 2011/0262377 A1.

An embodiment comprises an olfactory-active substance, such as a fragrance, an artificial tree to serve as a physical support, a reservoir at the top of said tree for holding the olfactory-active substance, and a series of transport elements connected to the reservoir that are capable of moving the olfactory-active substance within and throughout the tree. The interior or exterior of the trunk may be lined with a plurality of transport elements, such as but not limited to tubes or strips of wicking material, for the purpose of moving the olfactory-active substance down from the reservoir at the top of the tree and throughout the trunk of the tree. Transport elements may branch out from the trunk to form a network of transport elements found throughout the tree including among the needles and branches. Transport elements may be placed into hollow needles, or if the transport element is of suitable material, it may be shaped and dyed in order to simulate a needle itself. Transport elements may be comprised of a suitable size, substance, and matrix such that the olfactory-active substance may be drawn down through the trunk by gravity while maintaining the ability to emit the scent of the olfactory-active substance.

Transport elements may be provided individually or in groups of two or more, and may be inside or outside of the trunk. Transport elements may be provided in any shape, for example, as a tube or a sleeve. Transport elements may optionally comprise dyes, coating compositions, holder elements or attachment elements as necessary.

Transport elements of the present invention comprise a structural component suitable for transporting the olfactory-active substance from the reservoir to distal points on the tree. Transport elements may be comprised of tubes with minuscule opens or mesh-like sections for the purpose of allowing fragrance molecules to emit from the olfactory active substance within. The transport elements may be comprised of permeable material, such as felt, paper, string, or cloth that allows fragrance to seep out of the olfactory-active substance therein or the transport elements may be comprised of any other suitably porous material or pattern for the purpose of emitting fragrance. Alternatively, transport elements may comprise closed tubing or similar structures that are only open at the termini. The release from the termini may be controlled by the size of the opening that is present, a valve, or a fragrance-emitting device capable of holding the olfactory-active substance and emitting the associated fragrance. The fragrance emitting device may be, but is not limited to, a sponge or sponge-like material, a pulp or paper matrix, a plastic, or any other suitable substrate for the purpose of controlling release from the transport elements while emitting fragrance. The fragrance-emitting devices, which may be made from a matrix material composition, may contain within or on the device a matrix material comprising an olfactory-active substance.

The present invention comprises compositions used in making a fragrance-emitting device. A structural component may comprise one or more matrix material compositions. A matrix material composition may comprise natural and synthetic pulp compositions, or pulp compositions combined with other products, including but not limited to paper, cellulose, cellulose acetate, pulp lap, cotton linters, biological plant-derived materials (from living plants), synthesized pulp compositions, and mixed pulps. A matrix material composition may comprise additives, materials that provide strength, rigidity or structure to a matrix material composition. For example, a composition may comprise nanomaterials as additives. A fragrance-emitting device of the present invention may be referred to as a pulp article, indicating that the article comprises a matrix material composition which was made from a pulp composition of the present invention.

The matrix material used within the fragrance-emitting device may be a variety of materials including pulps, papers, sponges, and felts of different densities and porosities as well as those materials incorporated herein by reference from published US patent application US 2011/0262377 A1.

An embodiment of the present invention comprises incorporating an olfactory-active substance into the physical structure of an artificial tree. Any solid component of the artificial tree that requires shaping or forging may have an olfactory-active substance incorporated within it during the fabrication process. Material components which may be suitable for this process include all types of synthetic plastics, PVCs, polyethylene, and resins that may be used for forming the trunk, stand, needles, leaves, or branches of the artificial tree. Olfactory-active substances as defined herein can include but is not limited to fragrances, repellants, odor-eliminating compounds, aromatherapy compounds, natural oils, water-based scents, odor neutralizing compounds, and cyclodextrins. Before the solidification or shaping of the components of the tree or during the molding or extrusion processes, a suitable amount of olfactory-active substance may be added to the mixture such that the final product emits a preferred scent or aroma. An aspect of the present invention comprises an artificial tree wherein the trunk of the tree may be made from a plastic or PVC comprising an olfactory-active substance. An aspect of the present invention comprises an artificial tree wherein the branches of the tree may be made from a plastic or PVC comprising an olfactory-active substance. An aspect of the present invention comprises an artificial tree wherein the leaves or needles of the tree may be made from a plastic, a polyethylene, or PVC comprising an olfactory-active substance. An aspect of the present invention comprises an artificial tree wherein the stand of the tree may be made from a plastic or PVC comprising an olfactory-active substance. Material components that have been amalgamated with the olfactory-active substance can then be used as the physical pieces of an artificial tree.

A coating applied to a matrix material or to a structural component of a tree of the present invention may function as a barrier and thus retard release of an olfactory-active substance from the matrix material or structural component of the tree. Alternatively, a coating may be used to provide the olfactory-active substance so as to add fragrance to the coated portion of the tree. As used herein, a coating, applying a coating and a coating composition refer to providing this barrier function to a matrix material or a structural component. Coating of a matrix material may occur before the matrix material is formed into the structural component, such as a tree, trunk, branch or needle, or may occur after the structural component is formed. A coating applied to a matrix material may be in a pattern, such as a grid, applied to cover one entire surface, or may be randomly applied. An olfactory-active composition may be encapsulated and grafted to a matrix material before or after extrusion, shaping, forming, molding, or cutting a matrix material to form a tree component, such as a trunk, branch, or needle, or an adjunct scent device.

Coating compositions may comprise waxes, including but not limited to soy waxes, paraffins, and bees waxes. The wax may be coated onto a matrix material or a structural component using a flood coating method, wherein a wax is diluted in a solvent, such as a volatile solvent, and all outer surfaces are uniformly coated, for example, with a 1-10 micron layer of wax. A wax may be diluted in pentane. The coating may coat outer surfaces, including an outer surface that forms the inner surface that would be next to a core material. For example, in a rod shaped article shaped like a drinking straw, both the outer surface and the inner bore surface may be coated with a material, such as a wax. Waxes contemplated by the present invention comprise waxes known to those skilled in the art. A hydrophobic or hydrophilic solvent may be used in a coating composition.

Microcrystalline waxes differ from refined paraffin wax in that the crystal structure is more branched and the carbon chains are longer. These waxes are tougher, more flexible and have higher tensile strengths and melting points. They are also more adhesive, and they bind solvents, oil, etc., and thus prevent the sweating-out of compositions. Polyethylene waxes are manufactured from low molecular weight, high-density raw materials, designed to give the particular performance characteristics required by industry.

Waxes that may be used as coating compositions may comprise waxes that soften or melt at a temperature about 150 F, or in excess of 200 F. For example, the High-Melt Straight Paraffin product from International Group Inc, IGI 1260-SLAB has a melting point of 163° F. Synthetic hydrocarbon waxes may have high melting points—e.g. 210° F. Coatings with high melting points, such as above 250° F., above 400° F. or higher, are contemplated by the present invention.

Coating compositions may comprise acrylates. For example, coating compositions may be made with polyester polymers, such as polylactide, polyglycolide or polycaprolactone, or a polyester copolymer selected from poly(lactide/glycolide) acid (PLGA) or poly(lactid-co-ε-caprolact-one) (PLCL), alkyl- or alkoxyalkyl-2-cyanoacrylates such as n-butyl-2-cyanoacrylate or 2-methoxybutyl-2-cyanoacrylate, crosslinked cyanoacrylate, polylactic acid, polyglycolic acid, lactic-glycolic acid copolymers, polycaprolactone, lactic acid-caprolactone copolymers, poly-3-hydroxybutyric acid, polyorthoesters, polyalkyl acrylates, copolymers of alkylacrylate and vinyl acetate, polyalkyl methacrylates, and copolymers of alkyl methacrylates and butadiene; and plasticizers such as dioctyl phthalate, dimethyl sebacate, trethyl phosphate, tri(2-ethylhexy)phosphate, tri(p-cresyl)phosphate, glyceryl triacetate, glyceryl tributyrate, diethyl sebacate, dioctyl adipate, isopropyl myristate, butyl stearate, lauric acid, dibutyl phthalate, trioctyl trimellitate, and dioctyl glutarate.

Coating compositions may include polymers or copolymers containing, for example, caprolactone, poly-β-hydroxybutyrate, delta-valerolactone, as well as polyvinylpyrrolidone, polyamides, gelatin, albumin, proteins, collagen, poly(orthoesters), poly(anhydrides), poly(α-cyanoacrylates), poly(dihydropyrans), poly(acetals), poly(phosphazenes), poly(urethanes), poly(dioxinones), cellulose, proteins and starches. A coating contemplated by the present invention comprises Krylon® Triple-Thick crystal clear glaze, commercially available from Krylon Products Group, Cleveland, Ohio.

An embodiment of the present invention comprises covering or treating an artificial tree with a coating that comprises an olfactory-active substance, so that at least a portion of the tree is coated. Before the coating is applied to the artificial tree, the coating may be mixed, admixed, or, in some way known to those skilled in the art, combined with a suitable amount of olfactory-active substance such that the coated tree emits a fragrance. Alternatively, the olfactory-active compound may be applied concurrently with the coating material or after the coating material has been applied to the tree.

An embodiment of the present invention comprises the material components of an artificial tree which may be made of substances other than those normally associated with artificial trees such as wire, plastics, PVCs, and resins. Specific portions of the tree, such as all or a suitable portion of the needles, may be constructed of a matrix suitable for holding an olfactory-active substrate. Such matrices may be composed of materials including but are not limited to pulp, paper, felt, cloth, sponge, or foam as well as all of those matrices included herein by reference from published US patent application US 2011/0262377 A1. Matrices should be suitably porous for the purpose of emitting fragrance and may be shaped or dyed as necessary in order to blend into the tree.

An embodiment of the present invention comprises an artificial tree that is treated with an olfactory-active substance whereby the tree is covered with the olfactory-active compound and the tree itself is the fragrance-emitting device. Those areas that were treated with the olfactory-active substance may be coated with a desirable coating material such that the fragrance given off by the olfactory-active substance is maintained and released over a desired amount of time. The coating may be applied over the entire area of the tree, over specific portions of the tree, in specific patterns, or randomly.

An embodiment of the present invention comprises an olfactory-active substance, such as a fragrance, a suitable fragrance-emitting device for containing said olfactory-active substance, and an artificial tree to serve as a physical support, wherein the tree comprises a trunk, from which a plurality of branches is outwardly extending. The trunk may be hollow and comprised with a pattern of openings such as slits or holes such that ambient air can pass freely through them or the trunk may be solid. The interior or exterior of the trunk, as well as the leaves or needles or branches, may be lined with a plurality of housing devices capable of holding fragrance-emitting devices wherein fragrance-emitting devices are capable of emitting scent or aroma from the olfactory-active substance contained within them. The fragrance-emitting devices may be comprised of a suitable size, substance, and matrix and they may be attached to a tree by any suitable means such as hooks or snaps whereby they could be periodically replaced if necessary.

The housing devices may be provided individually or in groups of two or more, and may serve as receptacles for fragrance-emitting devices. Housing devices may include but are not limited to attachment elements including hooks, notches, cups, pockets, or ties. Housing devices may be comprised of any suitable material and of appropriate size and shape for holding the fragrance-emitting devices in place. For example, housing devices may comprise plastic hooks that are physically attached to the trunk wherein a fragrance-emitting device may be hung from said hooks. In another example, housing devices may comprise a pocket attached to the trunk wherein a fragrance-emitting device may be placed within the pocket. In an example, housing devices may comprise mesh pockets that are attached to the trunk wherein a fragrance-emitting device may be placed within the mesh pocket.

An aspect of the present invention comprises scent devices that may be associated with a tree disclosed herein. For example, a scent device or fragrance-emitting device may be placed anywhere on a tree or near a tree. A scent device may have any shape, such as a ball, a bird, a butterfly, or other animate or inanimate object shape. The scent device may emit a fragrance by one or more methods as described herein. The scent device may contain a matrix comprising an olfactory-active substance, or the entire device or a portion of the device may be made from a material comprising an olfactory-active substance, or the device may be coated with a fragrance-emitting coating. The scent device may further comprise one or more of a light, a heating element or a fan.

A fragrance-emitting device of the present invention comprises a structural component which is made from a matrix material composition, and may contain an olfactory-active substance. A fragrance-emitting device may comprise a gel packet or a matrix infused with olfactory-active substance. A fragrance-emitting device may be any size or shape such as a rod-like shape or spherical. For example, the fragrance emitting device may be Scentscicles or Scentstems. A fragrance-emitting device may be coated, dyed, or further treated in forming the present invention.

The present invention comprises compositions used in making a fragrance-emitting device. A structural component may comprise one or more matrix material compositions. A matrix material composition may comprise natural and synthetic pulp compositions, or pulp compositions combined with other products, including but not limited to paper, cellulose, cellulose acetate, pulp lap, cotton linters, biological plant-derived materials (from living plants), synthesized pulp compositions, and mixed pulps. A matrix material composition may comprise additives, materials that provide strength, rigidity or structure to a matrix material composition. For example, a composition may comprise nanomaterials as additives.

The matrix material used within the fragrance-emitting device may be a variety of materials including pulps and felts of different densities and porosities as well as those materials incorporated herein by reference from published US patent application US 2011/0262377 A1.

Optionally, the trunk may contain a fan or an equivalent device for moving air through the trunk of the tree for the purpose of further dispersing the fragrance. The fan may be electrically powered by any suitable means which may include but is not limited to battery power, solar or another type of light cell, or connection to alternating current (wall plug). The fan may be located at the bottom, middle, or top of a tree, and it may be oriented such that it is forcing air up or down. Optionally, the artificial tree may comprise one or more heating elements to heat the olfactory-active substance so as to release fragrance molecules. A heating element and fan may be provided together or in close proximity. A heating element may be electrically powered by any suitable means which may include but is not limited to battery power, solar or another type of light cell, or connection to alternating current (wall plug). A heating element may be located at the bottom, middle, or top of a tree, or found in several locations on or within the tree.

An embodiment of the present invention comprises an olfactory-active substance, such as a fragrance, a suitable reservoir for containing said olfactory-active substance, a pump mechanism or device, a series of transport elements running throughout the tree that are connected to the reservoir and the pump, and an artificial tree to serve as a physical support, wherein the tree comprises a trunk, from which a plurality of branches is outwardly extending. The interior or exterior of the trunk or the branches may be lined with a plurality of transport elements or matrices capable of emitting scent or aroma from the olfactory-active substance contained within them. Transport elements are comprised of a suitable size, substance, and matrix and they may be attached anywhere on, in or near a tree, for example, to the interior of trunk by any suitable means such as hooks or snaps.

A pump system may be constructed such that transport elements, such as tubes, are connected from the reservoir to the pump and then further dispersed throughout the tree. The pump itself may be electrically powered by a suitable power source such as but not limited to batteries, a solar or other type of light cell, or connection to alternating current (a wall plug). Pumps of this nature are known by those skilled in the art. The tubes may branch at multiple points, creating an internal network of tubes to run throughout the trunk and out onto the branches. All of the tubes may ultimately be attached to a fragrance-emitting device capable of emitting the scent or aroma of the olfactory-active substance. A fragrance-emitting device can comprise the needles of the plant being made of a suitable material or containing a suitable matrix. The fragrance-emitting device may be large and disperse like a web or small and consolidated at the end of the tube. The article may be comprised of a spongy material or a wicking material or any other suitable matrix like those incorporated herein by reference to the published US patent application US 2011/0262377 A1.

The transport elements may be provided individually or in groups of two or more, and may be inside or outside of the trunk. Transport elements may be provided in any shape, for example, as a tube or a sleeve. Transport elements may optionally comprise dyes, coating compositions, holder elements or attachment elements as necessary.

Transport elements of the present invention comprise a structural component suitable for transporting the olfactory-active substance from the reservoir to distal points on the tree. The transport elements may be comprised of tubes with minuscule opens or mesh-like sections for the purpose of allowing fragrance to emit from the olfactory active substance within. The transport elements may be comprised of permeable material, such as felt, paper, string, or cloth that allows fragrance to seep out of the olfactory-active substance therein or the transport elements may be comprised of any other suitably porous material or pattern for the purpose of emitting fragrance. Alternatively, transport elements may comprise closed tubing or similar structure that is only open at the termini. Release of olfactory-active substance from the termini may be controlled by the size of the hole that is present, a valve, or a fragrance-emitting device capable of holding the olfactory-active substance and emitting the associated fragrance. The fragrance-emitting device may be but is not limited to a sponge or sponge-like material, a pulp or paper matrix, or any other suitable substrate for the purpose of controlling release from the transportation elements while emitting fragrance. The fragrance-emitting devices, which may be made from a matrix material composition, may contain within them an olfactory-active substance.

An embodiment of the present invention comprises an artificial tree and a scented strand that may be or may not be electrically powered. A scent strand may be composed of a suitable substrate or matrix for containing an olfactory-active substance. A scent strand may comprise a cable or strand surrounded by a sleeve. A sleeve may be a composition of suitable matrix to contain an olfactory-active substance. Alternatively a sleeve may be treated with an olfactory-active substance, coated with a mixture of coating and olfactory-active substance, or otherwise contain the property of fragrance.

A scented strand may be a capillary or transport element described herein. A scented strand may release scent near a light provided on the tree, for example, a Christmas light in a strand of Christmas lights. The scented strand may be a strand woven with the Christmas light strand, or may be attached or unattached to the light strand. The heat of the lights may aid in dispersing the fragrance from the scented strand. Alternatively, the light strand may be coated with a scented coating that releases an aroma when the lights are on and heated.

A cable may comprise lights such as Christmas lights or it may be designed to blend in with the tree. An electrical cable may be powered by any suitable power source, including but not limited to battery power, solar or other types of light cells, or connection to alternating current (a wall plug). An electrical cable may be capable of heating up when plugged in or powered up and the effect of this heating may be to facilitate the fragrance-emitting properties of the sleeve. An electrical cable may be connected to a timer, such that the cable may warm at routine intervals, for a specified amount of time, continuously, or randomly. An electrical cable may also be connected to a power supply regulator such that the electrical current flowing through the cable could be dialed up or down. A regulator may allow one to adjust the intensity of the fragrance-emitting facilitation by altering the flow of the current through the cable. A regulator may have distinct settings, such as low, medium, and high, or a regulator may function on a continuum by turning a dial. Regulators of this nature are known in the art. Cables or strands disclosed herein may have connecting elements so that two or more cables or strands can be joined together, for example, in a head to tail arrangement, or circularized, or such connecting elements may be found anywhere along the cable or strand to make branching cables or strands. Such cables or strands may be made from materials disclosed herein, including, but not limited to, wicking materials, plastics or pulp or paper materials. Such cables or strands may be made from materials having an olfactory-active material incorporated into the material or on the material, or may be coated with fragrance-emitting coatings, or have wrappers or sleeves comprising one or more olfactory-active substances.

An embodiment of the present invention comprises scenting an artificial tree with attachment elements, such as a hook, a j-shaped article, an openable circle, or other attachment elements capable of attaching one article to another article, such as attaching an ornament to a tree, that have fragrance-emitting properties. Attachment elements may be used to attach other accessories to a tree or they may be used independently. Attachment elements may be comprised of a suitable material for containing an olfactory-active substance, such as but not limited to paper, plastic, foam, plastic or sponge. Alternatively, attachment elements may be treated with an olfactory-active compound such that it is dried onto the surface, treated with an olfactory-active compound and then coated with a suitable coating to control the release of fragrance, or treated concurrently with an olfactory-active compound and a coating.

An attachment element may comprise one or more matrix material compositions. A matrix material composition may comprise plastics, natural and synthetic pulp compositions, or pulp compositions combined with other products, including but not limited to paper, cellulose, cellulose acetate, pulp lap, cotton linters, biological plant-derived materials (from living plants), synthesized pulp compositions, and mixed pulps. A matrix material composition may comprise additives, materials that provide strength, rigidity or structure to a matrix material composition. For example, a composition may comprise nanomaterials as additives.

An attachment element may be disposable and replaced with new attachment elements whenever fragrance emission is exhausted. For instance, in one example scented attachment elements may be used to hang decorations on an artificial Christmas tree, and said attachment elements may be replaced annually. Alternatively, attachment elements may be coated or constructed in such fashion that there is no need for disposal or replacement.

An embodiment of the present invention comprises scenting an artificial tree with attachable or proximal scented accessories. Scented accessories may be placed directly into or on to the artificial tree. For example, scented decorative bulbs or balls may be hung from an artificial tree, in particular a Christmas tree. For example, in a palm tree, an accessory item, such as a scented bird, may be placed on the trunk or among the fronds. Alternatively, scented accessories may be placed in the proximity of an artificial tree such that scent is provided. For example, a decorative box designed to look like a Christmas present may contain a fragrance-emitting device or devices.

Scented accessories may be comprised of a suitable material for containing an olfactory-active substance, such as but not limited to paper, plastic, foam, or sponge such that the accessory itself is a fragrance-emitting device. Alternatively, scented accessories may be treated with an olfactory-active compound such that it is dried onto the surface, treated with an olfactory-active compound and then coated with a suitable coating to control the release of fragrance, or treated concurrently with an olfactory-active compound and a coating.

A scented accessory may comprise structural component of one or more matrix material compositions. A matrix material composition may comprise natural and synthetic pulp compositions, or pulp compositions combined with other products, including but not limited to paper, cellulose, cellulose acetate, pulp lap, cotton linters, biological plant-derived materials (from living plants), synthesized pulp compositions, and mixed pulps. A matrix material composition may comprise additives, materials that provide strength, rigidity or structure to a matrix material composition. For example, a composition may comprise nanomaterials as additives.

The matrix material used within the scented accessory may be a variety of materials including pulps and papers of different densities and porosities as well as those materials incorporated herein by reference from published US patent application US 2011/0262377 A1.

A scented accessory may be disposable and replaced whenever fragrance emission is exhausted.

Alternatively, scented accessories may not be fragrance-emitting devices in and of themselves, but rather comprise a housing unit for fragrance-emitting devices. The scented accessory may be designed such that a fragrance-emitting device may be held within it and the release of the fragrance may be controlled over time. For example, a decorative box that resembles a Christmas present may house Scentsicles or Scentstems wherein the fragrance is continually released for an extended period of time.

A scented accessory may comprise a scented light bulb cover. The light bulb cover may be placed over standard lights, such as the bulbs of a strand of Christmas lights, or any occasion.

An embodiment of the present invention comprises an artificial tree that is stored and/or sold in fragrant packaging. Packaging may include but is not limited to a box, bag, or other suitable container for the purpose of transporting an artificial tree. Packaging may be made from cardboard, foam board, pressed paper, wood, cloth, or other suitable materials. Packaging further comprises any packing or stuffing materials necessary for maintaining the integrity and safety of an artificial tree.

Packaging itself may be treated with olfactory-active substances in such a way as to serve as a fragrance-emitting device. Interior packing or stuffing, which may include but is not limited to Styrofoam, paper, plastic, cardboard, or foam, may also be treated with olfactory-active substances such that it is able to emit fragrance.

Materials used at the point of sale of an artificial tree may be scented with olfactory-active substances and materials as described herein. The point of sale materials, such as boxes or stands may be made from materials incorporating olfactory-active substances and may or may not be coated to control release of fragrance. A point of sale material may be coated with a scented coating. A point of sale material may have a reservoir, capillaries or transport elements as described herein. Optionally, separate scent dispensing devices may be attached to or adjacent to the point of sale materials. Such materials may optionally comprise fans or heaters to aid in scent dispersion.

An embodiment of the present invention comprises an artificial tree with a scent dispensing device.

The tree comprises a trunk, from which a plurality of branches is outwardly extending. The plurality of branches may generally extend out from the upper portion of the trunk, while the lower portion may be generally devoid of branches. The trunk may be generally columnar in shape and generally vertically oriented along a longitudinal axis. The trunk may be hollow and comprised with a pattern of openings such as slits or holes such that ambient air can pass freely through them. Alternatively, the trunk may be solid.

A scent dispensing device comprises a housing unit for holding a fragrance-emitting insert and a heating element wherein the heat produced by the heating element contacts the fragrance-emitting insert sufficiently to facilitate the emission of fragrance.

A scent dispensing device can be located at any point in the tree or proximally to the tree. In an aspect of the present invention, the scent dispensing device is located in the bottom portion of a hollow trunk. In an aspect of the present invention, the scent dispensing device is located in the middle portion of a hollow trunk. In an aspect of the present invention, the scent dispensing device is located in the upper portion of a hollow trunk. In an aspect of the present invention, the scent dispensing device is located in the stand. In an aspect of the present invention, the scent dispensing device is connected to the outside of the trunk such that it is hidden by the branches. In an aspect of the present invention, the scent dispensing device is located proximally to the tree. In an aspect of the present invention, the scent dispensing device is located proximally to the tree and within a decorative accessory, for example, within a box that resembles a Christmas present.

A housing unit may be composed of a suitable material to withstand the heat produced by the heating element. It may be of suitable size and shape to house the fragrance emitting insert which may include but is not limited to Scentsicles, Scentstems, gel inserts, or other fragrance-emitting devices. Embodiments of Scentsicles and Scentstems are disclosed and/or claimed in U.S. patent application Ser. No. 11/571,240, which is herein incorporated in its entirety.

A heating element comprises a means of producing sufficient heat to facilitate the emission of fragrance. A heating element is any conventional heating device that provides sufficient heat to facilitate emission of fragrance. In an aspect of the present invention, the heating element is an electrode. In an aspect of the present invention, the heating element is a heat lamp. In an aspect of the present invention, the heating element is a light.

A heating element may be powered by any suitable power source which may include but is not limited to battery power, solar or other light cells, or connection to alternating current (a wall plug). Optionally, the heating element may be connected to a timer such that the activation of the heating element is controlled by said timer. A timer may allow the heating element to be turned at routine intervals, for a specified amount of time, constitutively, or randomly. The timer may be powered by any suitable power source which may include but is not limited to battery power, solar or other light cells, or connection to alternating current (a wall plug).

Optionally, the scent dispersing device comprises a fan. The fan may be powered by any suitable power source which may include but is not limited to battery power, solar or other light cells, or connection to alternating current (a wall plug). The fan may also be connected to the timer such that it is activated in concert with the heating element for additional perfusion of fragrance. The fan may be located at the bottom, middle, or top of a tree or it may be located adjacent to the scent dispensing device. The fan may be oriented such that it forces air past the scent dispensing device.

That which is claimed is:

1-16. (canceled)

17. A method of scenting an artificial tree using at least one scented accessory.

18-20. (canceled)

21. The method of claim 17, wherein the at least one scented accessory comprises a matrix material composition comprising an olfactory-active substance.

22. The method of claim 21, wherein the matrix material composition is a pulp composition.

23. The method of claim 17, wherein the at least one scented accessory is treated with an olfactory-active compound.

24. The method of claim 22, wherein the at least one scented accessory is coated to control fragrance release.

25. The method of claim 17, wherein the at least one scented accessory comprises a ball, bird, butterfly, holiday ornament, or gift box shape.

26. The method of claim 17, wherein the at least one scented accessory comprises a light, heating element, or fan.

27. A scented accessory for scenting an artificial tree comprising:
an olfactory-active substance; and
a dispersal mechanism;
wherein the dispersal mechanism introduces a scent into ambient air.

28. The scented accessory of claim 27, wherein the scented accessory comprises a ball, bird, butterfly, holiday ornament, or gift box shape.

29. The scented accessory of claim 27, wherein the dispersal mechanism comprises a matrix material composition.

30. The scented accessory of claim 29, wherein the matrix material composition is a pulp composition.

31. The scented accessory of claim 27, wherein the dispersal mechanism comprises an olfactory-active substance emitting coating.

32. The scented accessory of claim 27, wherein the dispersal mechanism comprises a material impregnated with the olfactory-active substance.

33. The scented accessory of claim 27, further comprising a reservoir to hold the olfactory-active substance.

34. The scented accessory of claim 33, wherein the reservoir is refillable or replaceable.

35. The scented accessory of claim 27, wherein the dispersal mechanism is a capillary dispersal mechanism.

36. The scented accessory of claim 27, wherein the dispersal mechanism is a gravity-fed dispersal mechanism.

37. The scented accessory of claim 27, wherein the dispersal mechanism comprises a light, heating element, pump, or fan.

38. The scented accessory of claim 27, further comprising a power source.

39. The scented accessory of claim 38, wherein the power source comprises a battery, wall plug, or decorative lighting string.
